(19) United States
(12) Patent Application Publication
(10) Pub. No.: US 2015/0119945 A1
(43) Pub. Date: Apr. 30, 2015

(54) SET SCREW TO MINIMIZE SPLAYING IN PEDICLE SCREWS

(71) Applicant: Orthopaedic International, Inc., Cabuyao (PH)

(72) Inventors: Jude L. Sasing, Quezon City (PH); Mark Dranreb O. Royo, Cabuyao (PH)

(21) Appl. No.: 14/529,842

(22) Filed: Oct. 31, 2014

Related U.S. Application Data
(60) Provisional application No. 61/898,223, filed on Oct. 31, 2013.

(51) Int. Cl. A61B 17/86

(52) U.S. Cl. CPC A61B 17/8605
USPC 606/308; 29/525.11; 29/525.05

(57) ABSTRACT

A bone screw assembly including a bone screw that has a head portion with a base, a pair of arms extending upwardly from the base, and an internally threaded slot defined by the pair of arms, and a screw portion extending downwardly from the base of the head portion. The assembly further includes a set screw having a body with a threaded outer surface and a distal tip, wherein the distal tip comprises a truncated taper portion extending from a base to generally flat distal end. The threaded outer surface of the set screw is engageable within the internally threaded slot of the head portion of the bone screw.

### CROSS-REFERENCE TO RELATED APPLICATIONS

This application claims the benefit of U.S. Provisional Patent Application No. 61/898,223, filed Oct. 31, 2013 and titled “SET SCREW TO PREVENT SPLAYING IN PEDICLE SCREWS”, which application is incorporated herein by reference in its entirety.

### TECHNICAL FIELD

The present invention relates to set screws used in conjunction with pedicle screws for spinal stabilization. In particular, the present invention provides for a set screw tip configuration that minimizes or prevents the splaying of the head of pedicle screws.

### BACKGROUND

Fractures and injuries of the vertebral column are usually treated with pedicle screws in conjunction with a spinal rod to stabilized the injury and fuse the vertebrae in the affected area. The pedicle screws are usually equipped with a head portion with a slot to accommodate the spinal rod. The spinal rod connects the pedicle screws at different levels of the spine. A set screw is used to fasten each pedicle screw to the spinal rod to create an implant construct that promotes fusion of the affected vertebrae. It is important that a strong interconnection is achieved between the pedicle screw and spinal rod in order to have a stable implant construct.

In order to achieve the required interconnection strength between the pedicle screw and spinal rod, the set screw is tightened to a certain torque. However, since the head of the pedicle screw has a slot to accommodate the spinal rod, the head splays during tightening of the set screw. The higher the tightening torque, the more the head splays. If the splaying becomes excessive, the set screw can jump out of place or become disengaged from the pedicle screw head, resulting in a weakened implant construct. The present invention allows for an effective way of fastening the pedicle screw and the spinal rod, while minimizing or eliminating the chance of the set screw from jumping out of place or becoming disengaged.

### SUMMARY

Bone screws and assemblies that include bone screws that are assembled with an elongated rod are provided for use in spinal stabilization systems. The bone screws include a head portion with a slot for receiving the rod, and a set screw inserted into the slot to secure the rod in the slot. In order to minimize or eliminate splaying of the head during tightening of the set screw while minimizing or eliminating set screw disengagement, set screws are configured to reduce the area of rod contact to reduce torsional friction while increasing clamping force, and to reduce the torque required for adequate tightening of the set screw. Embodiments of the invention can use a break-off head on the set screw to further control the tightening torque, and can include the use of modified square threads.

In accordance with an aspect of the invention, a bone screw assembly is provided that includes a bone screw and a set screw. The bone screw includes a head portion comprising a base, a pair of arms extending upwardly from the base, and an internally threaded slot defined by the pair of arms, and a screw portion extending downwardly from the base of the head portion. The set screw includes a body having a threaded outer surface and a distal tip, wherein the distal tip comprises a truncated taper portion extending from a base to generally flat distal end, wherein the threaded outer surface of the set screw is engageable within the internally threaded slot of the head portion of the bone screw. The bone screw assembly can be combined with a rod member, wherein the slot comprises a generally U-shaped lower surface that is sized and shaped to accept an outer surface of the rod member, and wherein the flat distal end of the set screw is engageable with the outer surface of the rod member. The base of the truncated portion can include an outer diameter that is greater than an outer diameter of the generally flat distal end. The set screw includes a minor diameter and a major diameter, wherein the truncated taper portion of an embodiment includes a base diameter that is equal to or smaller than the minor diameter of the set screw. The flat distal end of the truncated taper portion of the set screw can be a solid surface, and the set screw can further include a break-away portion extending from a proximal end of the body that is opposite the distal tip of the body.

In accordance with another aspect of the invention, a set screw is provided that is engageable with a head portion of a bone screw of a spinal bone screw assembly, the set screw comprising a body having a threaded outer surface and a distal tip, wherein the distal tip comprises a truncated taper portion extending from a base to a generally flat distal end, and wherein the base of the truncated taper portion comprises an outer diameter that is smaller than an outer diameter of the generally flat distal end. The set screw can further include a break-away portion extending from a proximal end of the body that is opposite the distal tip of the body, wherein the break-away portion is detachable from the body upon application of a predetermined amount of force, and the flat distal end of the truncated taper portion can be a solid surface.

In accordance with another aspect of the invention, a method is provided for assembling a bone screw assembly, including the steps of: inserting an elongated rod into an internally threaded U-shaped slot of a bone screw, the bone screw comprising a head portion comprising a base, a pair of arms extending upwardly from the base, and the internally threaded U-shaped slot that is defined by the pair of arms, and a screw portion extending downwardly from the base of the head portion, and inserting a set screw into the U-shaped slot of the bone screw, wherein the set screw comprises a body having a threaded outer surface and a distal tip that comprises a truncated taper portion extending from a base to generally flat distal end, and a break-away portion extending from a proximal end of the body that is opposite the distal tip of the body. The method further includes translating the set screw toward the elongated rod by rotating the set screw to further engage the threaded outer surface of the set screw body with the internal threads of the U-shaped slot until the generally flat distal end of the distal tip contacts an outer surface of the elongated rod. The method can further include translating the set screw toward the elongated rod further includes rotating the set screw until the break-away portion detaches from the set screw body.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of the components of a prior art pedicle screw construct;

FIG. 2 is a perspective view of an assembled prior art pedicle screw construct;

FIG. 3a is a perspective view of a prior art set screw;

FIG. 3b is a side view of the prior art set screw of FIG. 3a;

FIG. 3c is a front view of the prior art set screw of FIG. 3a in contact with a spinal rod;

FIG. 4 is a side view of another prior art set screw;

FIG. 5a is a side view of yet another prior art set screw;

FIG. 5b is a perspective view of the prior art set screw of FIG. 5a;

FIG. 6a is a perspective view of a set screw of the present invention;

FIG. 6b is a side view of a set screw of the present invention; and

FIG. 6c is a front view of a set screw of the present invention as it can be included in an assembly that includes a pedicle screw and a spinal rod.

### DETAILED DESCRIPTION

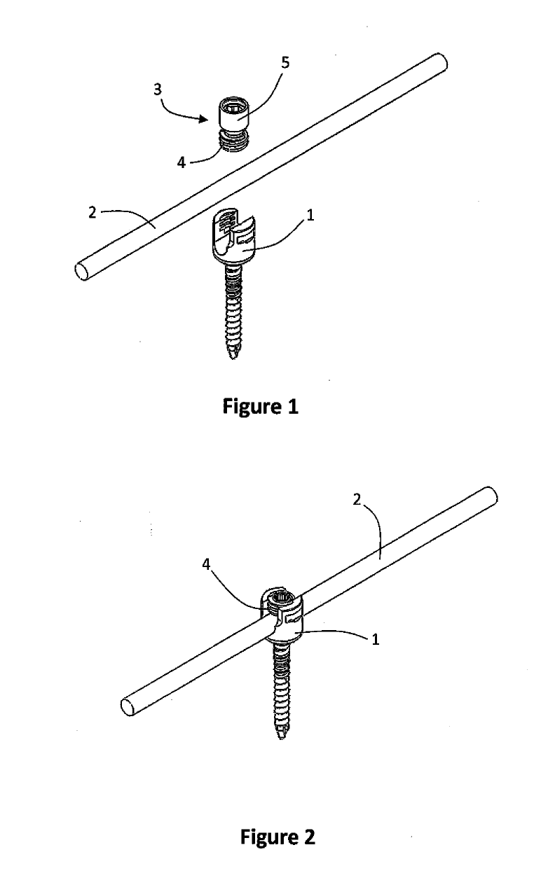

Referring now to FIGS. 1 and 2, there are shown the components of a prior art pedicle screw construct composed of a pedicle screw 1, a spinal rod 2, and set screw 3. The set screw 3 has a threaded portion 4 and it can also be equipped with a break off head 5 that breaks off when the required tightening torque is reached. FIG. 2 shows the pedicle screw construct of FIG. 1 fully assembled with the break off head removed and the set screw tightened to the right tightening torque.

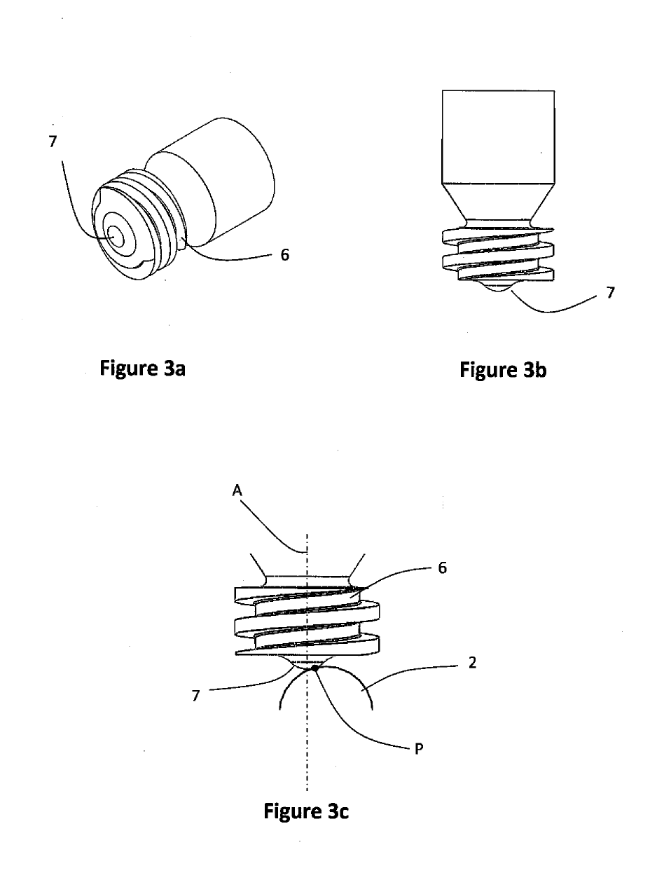

FIGS. 3a, 3b, and 3c show a prior art set screw 6 with a rounded tip 7. This set screw design provides a “point” contact between the set screw 6 and spinal rod 2. This allows efficient transformation of the tightening torque on the set screw 6 into a compressing force on spinal rod 2 as there is minimal frictional torque between the spinal rod 2 and rounded tip 7 due to the small contact area between set screw 6 and spinal rod 2. However, the design of set screw 6 has a distinct disadvantage that can result in excessive splaying of the pedicle screw head. Due to inherent inaccuracies in any manufacturing process, the contact point P between the spinal rod 2 and the set screw 6 may not be at the center of rounded tip 7, as shown in FIG. 3c. The offset of the contact point P from the axis A of rounded tip 7 will cause the set screw 6 to roll on spinal rod 2. This produces a tendency for set screw 6 to swerve or “walk” relative to spinal rod 2 as it is rotated during tightening. This can result in a sideways force on the pedicle screw head causing the pedicle screw head to splay, eventually resulting in disengagement of set screw 6 from the pedicle screw head. The propensity of set screw 6 to swerve will depend on the degree of manufacturing inaccuracies and on the eccentricity of rounded tip relative to spinal rod 2.

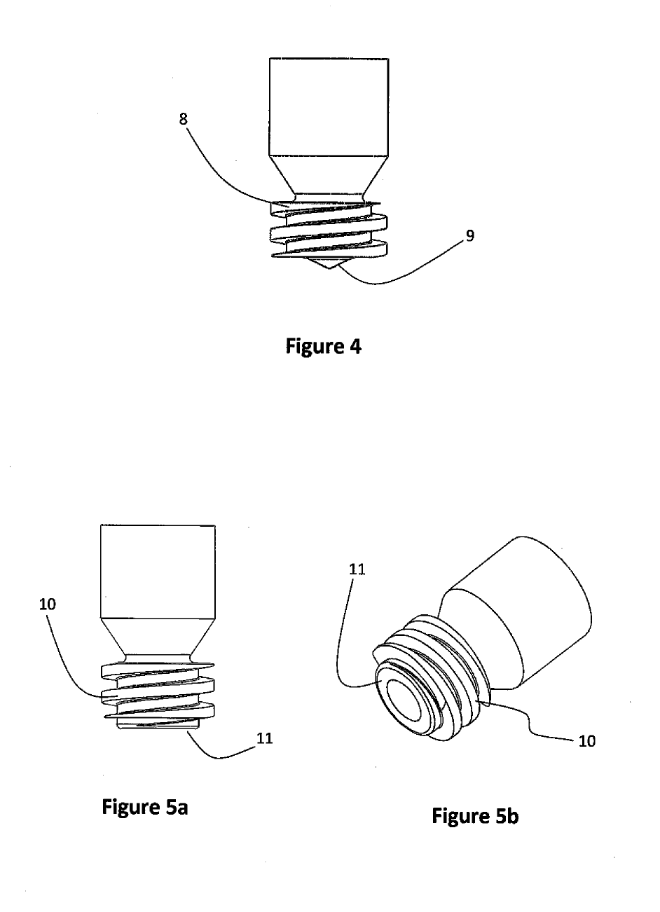

FIG. 4 shows another prior art set screw 8 with a cone tip 9. This type of set screw is efficient in transforming the tightening torque into a compressing force on spinal rod 2, and it does not swerve on spinal rod 2 as it is tightened because the contact point between the spinal rod 2 and the set screw is confined to be at the center of cone tip 9. However, cone tip 9 creates an indentation on spinal rod 2 during tightening that can significantly reduce the strength of spinal rod 2.

FIGS. 5a and 5b show another prior art set screw 10 with a flat ring tip 11 that has a constant diameter along its length. The flat ring tip 11 does not provide the swerving problem of set screw 6, and it does not embed into spinal rod 2 like set screw 8. The flat ring tip 11 of set screw 10 will not swerve when tightened on spinal rod 2 because there are two opposing segments of flat ring tip 11 that come into contact with spinal rod 2. The contact forces at each contacting segment are in opposite directions when the set screw is rotated during tightening, and the sideway forces cancel out, preventing the set screw 10 from swerving relative to spinal rod 2. However, the flat ring tip 11 does not provide for an efficient conversion of tightening torque into axial force. The distance between the contact segments creates a frictional couple that opposes the tightening torque. A significant amount of torque is spent on neutralizing the frictional coupling instead of being converted into axial compressive force.

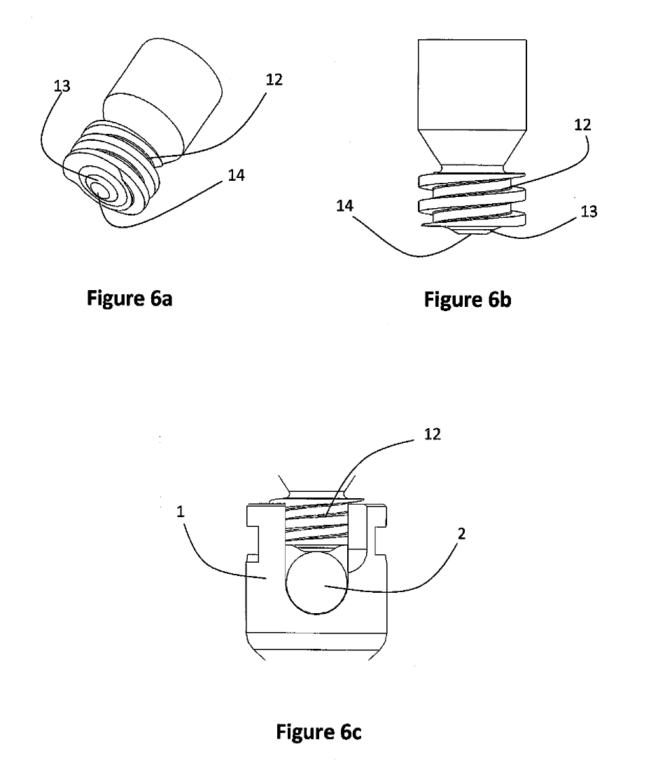

Referring now to FIGS. 6a, 6b, and 6c, embodiments of the present invention address the problem of swerving described above, while providing for an efficient conversion of tightening torque into axial compressive force. In particular, a set screw 12 is illustrated that includes a body having threaded outer surface extending along its length, and a distal tip 13. The distal tip 13 is configured as a truncated taper that extends from its base to a smaller, generally flat end 14. In an exemplary embodiment, the base of the truncated taper is the same size or smaller than the minor diameter of the set screw 12. Flat end 14 provides for multiple contact points at opposing sides of the axis of set screw 12, The distal tip 13 may include a radius between its base and the cylindrical body of the screw, or the transition may instead be relatively sharp or less rounded. In an exemplary embodiment, the flat end 14 is a solid, relatively planar surface that provides the desired contact with a rod, as is described below.

In another exemplary embodiment, the distal tip 13 can be cylindrical instead of tapered, in which case it will have generally the same diameter as the flat end 14. In this embodiment, the flat end 14 will have a diameter that is at least slightly smaller than the minor diameter of the set screw 12.

Like the contact forces of prior art set screw 10 described above, the contact forces at the tip of set screw 12 cancel out, preventing swerving of set screw 12 during tightening. However, unlike the flat ring tip 11 of set screw 10, the distances between contacting points on flat end 14 of the present invention are small, thereby reducing the magnitude of the frictional couple. Furthermore, unlike the pointed conical tip 9 of prior art set screw 8, flat end 14 of set screw 12 will not embed into a spinal rod, and therefore will not cause weakening of the spinal rod.

The set screw 12 is engageable with a pedicle screw or bone screw to provide a bone screw assembly, wherein the assembly includes engagement with an elongated rod when implanted in a patient. Such a pedicle screw can have a variety of different configurations, wherein set screw 12 is illustrated as being engaged with a portion of an exemplary pedicle screw 1 in FIG. 6c. As shown, pedicle screw 1 includes a head portion having a base and a pair of arms that are spaced from each other and extend upwardly from the base. The inner portion of these arms is threaded to provide an internally threaded slot, wherein the threaded outer surface of the set screw is engageable within the threads of this slot. The pedicle screw 1 further includes a screw portion (only a small portion of which is shown in FIG. 6c) that extends downwardly from the base of the head portion (i.e., in a generally opposite direction from the direction that the pair of arms extend from the base portion). The downwardly extending screw portion of the bone screw can be fixed relative to the head portion or can instead be moveably engaged with the head portion, as can be provided in configurations in which the extending screw portion includes a shaped head that cooperates with a corresponding opening within the head portion of a bone screw, for example.

The internal threads of the slot of the head portion and the corresponding threads of the outer surface of the set screw can have a number of configurations, including square threads, modified square threads (e.g., having a generally trapezoidal thread with a 10-degree included angle, or a standard 10-Degree Modified Square Thread), Acme threads, Butress threads, or V-threads. In accordance with the invention, the use of square or modified square threads provide for a stronger engagement between the inner and outer threads, even if the extending arms of the bone screw start to splay or move away from each other during the process of tightening the set screw. That is, as compared to V-threads with which the cross section of the contacting threads will decrease if the extending arms move away from each other, the use of square or modified square threads will provide for a more robust connection between the components, even if the arms splay away from each other. In an exemplary embodiment, the thread depth is relatively deep in order to maximize the amount of splaying that can be tolerated while still providing sufficient engagement of the set screw threads with the threads of the slot.

In additional embodiments of the invention, the set screw configurations described herein can be used with spinal implants other than pedicle screws, such as spinal hooks or other implant devices or configurations.

One method of using the bone screw assembly of the present invention is generally applied in surgery as follows. An elongated rod is selected to correct or maintain a certain relationship of adjacent vertebral bodies within a patient. This rod may be reconfigured on site for a particular patient, or may be preselected from available rods. In either case, the threaded portion of the bone screw is threaded into bone, such as the pedicle portion of a vertebral body, using a driving instrument such as a screwdriver. The elongated rod is then inserted into an internally threaded U-shaped slot of the bone screw, wherein the bone screw includes a head portion having a base, a pair of arms that extend upwardly from the base and define the U-shaped slot.

A set screw is then positioned within the internal threads of the extending arms and rotated to move the set screw toward the rod. As described above, the set screw includes a body having a threaded outer surface and a distal tip that comprises a truncated taper portion extending from a base to generally flat distal end, and a break-away portion extending from a proximal end of the body that is opposite the distal tip of the body. As the set screw is tightened, it pushes the elongated rod into the channel until the generally flat distal end of the distal tip contacts an outer surface of the elongated rod. The method may further include further rotation of the set screw until the break-away portion detaches from the set screw body.

Additional advantages and modifications of the present invention will readily occur to those skilled in the art in view of these teachings. The present invention in its broader aspects is not limited to the specific details, representative contrivances, and illustrative examples shown and described herein. Accordingly, various modifications may be made without departing from the spirit and scope of the general inventive concept.

What is claimed is:

1. A bone screw assembly comprising:
a bone screw comprising:
a head portion comprising a base, a pair of arms extending upwardly from the base, and an internally threaded slot defined by the pair of arms; and
a screw portion extending downwardly from the base of the head portion; and
a set screw comprising a body having a threaded outer surface and a distal tip, wherein the distal tip comprises a truncated taper portion extending from a base to generally flat distal end;
wherein the threaded outer surface of the set screw is engageable within the internally threaded slot of the head portion of the bone screw.

2. The bone screw assembly of claim 1, in combination with a rod member.

3. The combination of claim 2, wherein the slot comprises a generally U-shaped lower surface that is sized and shaped to accept an outer surface of the rod member, and wherein the flat distal end of the set screw is engageable with the outer surface of the rod member.

4. The bone screw assembly of claim 1, wherein the bone screw is a pedicle screw.

5. The bone screw assembly of claim 1, wherein the base of the truncated taper portion comprises an outer diameter that is greater than an outer diameter of the generally flat distal end.

6. The bone screw assembly of claim 1, wherein the set screw comprises a minor diameter and a major diameter, and wherein the truncated taper portion comprises a base diameter that is smaller than the minor diameter of the set screw.

7. The bone screw assembly of claim 1, wherein the set screw comprises a minor diameter and a major diameter, and wherein the truncated taper portion comprises a base diameter that is equal to the minor diameter of the set screw.

8. The bone screw assembly of claim 1, wherein the threaded outer surface of the set screw comprises modified square threads.

9. The bone screw assembly of claim 1, wherein the threaded outer surface of the set screw comprises square threads.

10. The bone screw assembly of claim 1, wherein the flat distal end of the truncated taper portion comprises a solid surface.

11. The bone screw assembly of claim 1, wherein the downwardly extending screw portion of the bone screw is fixed relative to the head portion.

12. The bone screw assembly of claim 1, wherein the downwardly extending screw portion of the bone screw comprises a shaped head moveably engaged with the base of the head portion.

13. The bone screw assembly of claim 1, wherein the set screw further comprises a break-away portion extending from a proximal end of the body that is opposite the distal tip of the body.

14. A set screw engageable with a head portion of a bone screw of a spinal bone screw assembly, the set screw comprising a body having a threaded outer surface and a distal tip, wherein the distal tip comprises a truncated taper portion extending from a base to a generally flat distal end, and wherein the base of the truncated taper portion comprises an outer diameter that is smaller than an outer diameter of the generally flat distal end.

15. The set screw of claim 14, further comprising a break-away portion extending from a proximal end of the body that is opposite the distal tip of the body, wherein the break-away portion is detachable from the body upon application of a predetermined amount of force.

16. The bone screw assembly of claim 14, wherein the flat distal end of the truncated taper portion comprises a solid surface.

17. A method of assembling a bone screw assembly, comprising the steps of:
inserting an elongated rod into an internally threaded U-shaped slot of a bone screw, the bone screw comprising;
a head portion comprising a base, a pair of arms extending upwardly from the base, and the internally threaded U-shaped slot that is defined by the pair of arms; and
a screw portion extending downwardly from the base of the head portion;
inserting a set screw into the U-shaped slot of the bone screw, wherein the set screw comprises:
a body having a threaded outer surface and a distal tip that comprises a truncated taper portion extending from a base to generally flat distal end; and
a break-away portion extending from a proximal end of the body that is opposite the distal tip of the body; and
translating the set screw toward the elongated rod by rotating the set screw to further engage the threaded outer surface of the set screw body with the internal threads of the U-shaped slot until the generally flat distal end of the distal tip contacts an outer surface of the elongated rod.

18. The method of claim 17, wherein the step of translating the set screw toward the elongated rod further includes rotating the set screw until the break-away portion detaches from the set screw body.
